(19) United States
(12) Patent Application Publication
(10) Pub. No.: US 2003/0190609 A1
(43) Pub. Date: Oct. 9, 2003

(54) Address/capture tags for flow-cytometery based minisequencing

(76) Inventors: P. Scott White, Los Alamos, NM (US); David C. Torney, Jemez Springs, NM (US)

Correspondence Address: Ray G. Wilson, Los Alamos National Laboratory, LC/IP, MS A187, Los Alamos, NM, 87545, US

(21) Appl. No.: 09/877,819

(22) Filed: Jun. 7, 2001

Related U.S. Application Data
(60) Provisional application No. 60/210,759, filed on Jun. 8, 2000.

(51) Int. Cl. C12Q 1/68; C12P 19/34

(52) U.S. Cl. 435/6; 435/91.2

(57) ABSTRACT

A method for generating address/capture tags for use in a sensitive and rapid flow-cytometry based assay for the multiplexed analysis of SNPs based on polymerase-mediated primer extension using microspheres as solid supports is described. Single-nucleotide polymorphisms (SNPs) are the most abundant type of human genetic variation. These variable sites are present at high density in the genome, making them powerful tools for mapping and diagnosing disease-related alleles. Subnanomolar concentrations of sample in small volumes (10 ml) can be analyzed at rates greater than one sample per minute, without a wash step. Genomic analysis using multiplexing microsphere arrays, enables the simultaneous analysis of dozens, and potentially hundreds of SNPs per sample. The method has been tested by genotyping the Glu69 variant from the HLA DPB1 locus, a SNP associated with chronic beryllium disease, as well as HLA DPA1 alleles.

### CROSS REFERENCES TO RELATED APPLICATIONS

This patent application claims the benefit of provisional application Serial No. 60/210,759 which was filed on Jun. 8, 2000.

### STATEMENT REGARDING FEDERAL RIGHTS

This invention was made with government support under Contract No. W-7405-ENG-36 awarded by the U.S. Department of Energy to The Regents of The University of California. The government has certain rights in the invention.

### FIELD OF THE INVENTION

The present invention relates generally to flow cytometry and, more particularly, to a method for generating address/capture tags for use in multiplexed flow-cytometry based assays.

### BACKGROUND OF THE INVENTION

Single nucleotide polymorphisms (SNPs) are the most frequent form of sequence variation among individuals (Cooper et al., 1985; Cooper and Krawczak, 1990). These sites are present at high density in the genome and are highly conserved, making them powerful tools for the mapping and diagnosis of disease-related alleles. As sequencing and mapping of the human genome near completion, the detection and analysis of SNPs for applications ranging from disease gene mapping to diagnostics will be a major objective for genome research (Schaffer and Hawkins, 1998; Brookes, 1999). Such applications could involve the screening of hundreds to hundreds of thousands of SNPs in thousands to tens of thousands of samples. There is at present a pressing need for SNP scoring methods that are robust, high throughput, and cost efficient.

A variety of assay configurations has been developed to score SNPs, including hybridization (Wang et al., 1998), ligation (Landegren et al., 1988), polymerase (Syvanen et al., 1990), and nuclease (Lee et al., 1993; Lyamichev et al., 1999). These assays have been adapted to a number of analysis platforms including electrophoresis (Pastinen et al., 1996), microplates (Tobe et al., 1996), mass spectrometry (Braun et al., 1997), and flat arrays (Wang et al., 1998). The ideal method for large-scale SNP scoring would use a robust assay chemistry combined with a flexible analysis plat-form, enabling the multiplexed analysis of many SNPs per sample in a highly automated manner.

Polymerase-mediated single-base extension of oligonucleotide primers, or minisequencing (Syvanen, 1999), has proven to be a straightforward and robust tool for SNP genotyping. This approach involves the annealing of a primer directly upstream of the site of interest and single-base extension by DNA polymerase using labeled dideoxynucleotide triphosphates (ddNTPs). Minisequencing is attractive because it requires only a single primer per SNP and uses polymerase specificity to interrogate base identity. Minisequencing assays have been adapted to a variety of assay platforms, including electrophoresis (Tully et al., 1996), microplates (Shumaker et al., 1996), oligonucleotide arrays (Pastinen et al., 1997), and homogeneous fluorescence assays (Chen and Kwok, 1999); however, each of these configurations has limitations that preclude high-throughput, multiplexed, and automated analysis.

Flow cytometry is capable of sensitive and quantitative fluorescence measurements of individual particles without the need to separate free from particle-bound label. Analysis rates are very high (hundreds to thousands of particles per second), and multiple fluorescence and light scatter signals can be detected simultaneously. These features make flow cytometry an extremely powerful analytical tool for the analysis of cellular and macromolecular assemblies (Nolan and Sklar, 1998).

Accordingly, it is an object of the present invention to provide a flow cytometric assay that combines minisequencing with genomic analysis using multiplexing microsphere arrays to enable high-throughput SNP scoring.

Another object of the invention is to provide a method for designing address/capture tags that are capable of high specificity in directing a specific assay to a specific microsphere population in a multiplexed assay.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

To achieve the foregoing and other objects, and in accordance with the purposes of the present invention, as embodied and broadly described herein, the method for identifying a set of sequences useful as address/capture tags includes: generating a chosen number of random DNA sequences having a chosen length; rejecting all reverse complementary sequences from the chosen number of random DNA sequences, the remaining sequences forming a first group of sequences; rejecting all sequences from the first group of sequences having common subsequences with a subsequence length greater than a chosen number of bases, the remaining sequences forming a second group of sequences; rejecting all sequences in the second group of sequences which can form stable hairpins, the remaining sequences forming a third group of sequences; and rejecting all sequences in the third group of sequences which can form stable dimers, the remaining sequences forming a fourth group of sequences; whereby a set of sequences is identified such that the sequences, if synthesized, would hybridize to their respective complements with a high degree of specificity.

Preferably, the method includes the steps of determining the melting temperature of each of sequence in the fourth group of sequences; rejecting all sequences that melt below a selected temperature, forming thereby a fifth group of sequences; and synthesizing a desired number of the sequences in the fifth group of sequences and complements thereof.

It is preferred that the selected melting temperature is between 50° C. and 70° C. and, more preferably, that the selected melting temperature is about 60° C.

It is also preferred that the method includes the step of rejecting all runs of bases greater than a chosen number of bases.

Benefits and advantages of the invention include a great increase in the number of assays that can be reliably performed simultaneously using flow cytometry.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1. Genotyping the Glu69 SNP of HLA DPB1 Exon II. Flow cytometry-based minisequencing was performed on SAP/Exol-treated PCR amplified genomic DNA as described in EXAMPLE protocols, and the results compared with those obtained from standard sequencing.

FIG. 2. Template Concentration and Cycle Number Dependence of Flow-Cytometry Based Minisequencing. Flow cytometry-based minisequencing was performed at various concentrations of template for 99 cycles (A), or at 1 nM template for various numbers of cycles (B).

FIG. 3. Multiplex Hybridization of Capture and Address Tags. Fluorescent capture oligos (25 nM) were hybridized to their respective address tags jmmobilized on microspheres, both individually and as a mixture, demonstrating the specificity of primer capture.

FIG. 4. DPA1 Exon 2 Sequence, SNP sites, and Primer Placement for Multiplexed

Minisequencing. Arrows show the direction and orientation of the DPA1 minisequencing primers for the underlined variable sites.

FIG. 5. Multiplex Genotyping of HLA DPA1 Alleles. A 350 bp fragment of exon 2 of DPA1 was amplified by PCR and subjected to 99 cycles of multiplexed minisequencing using primers described in Table 2. The primers were than captured onto address tag-bearing micro spheres and analyzed by flow cytometry. Presented are the biallelic genotyping results from four individual representative samples (A) and from an thirty samples (B) at the eight DPA1 sites.

### DETAILED DESCRIPTION OF THE INVENTION

Briefly, the present invention includes a method for the construction of a collection of double-stranded DNA sequences manifesting specificity of binding. Each double-strand thereof consists of a pair of reverse complementary sequences. Binding specificity means that under reasonable experimental conditions the binding between the single strands arising from the double-strand sequences of the collection will be restricted to the reverse complementary pairs of sequences. The motivation for generating such sequences is that they enables large numbers of experiments to be tagged with one strand from a sequence and localized, on microbeads as an example, using the other complementary strand.

First, many potential tag sequences (oligomers) are generated. These sequences are then investigated for interactions that appear stable enough to create problems in the assay. In practical terms, this is accomplished by calculating the stability of any unfavorable interaction and expressing it in terms of a ΔG value, then omitting those oligomers that are likely to be involved in such interactions. Finally, the abbreviated collection of potential sequences is sorted by predicted melting temperature (Tₘ) (Kaderali, 2001), and a subset is chosen that has a narrow window of Tₘ's. This facilitates efficient capture at a temperature that is equally favorable for all tags.

As an example, chosen complementary pairs will melt at 60° C., whereas all other pairs of strands will melt below 30° C. Between these two temperatures, the desired binding specificity is manifest. The selected sequences and their complementary sequences are then synthesized.

The microsphere-based flow cytometric minisequencing assay of the present invention was demonstrated for SNP analysis.

Reference will now be made in detail to the present preferred embodiments of the invention, examples of which are illustrated in the accompanying drawings.

A. Materials and Methods:

1. Oligonucleotides. The DNA oligonucleotides were synthesized on an automated Applied Biosystems Model 394 oligonucleotide synthesizer using biotin-phosphoramidite and biotin- or amino-amino CPG from Glen Research (Sterling, Va.) or ordered from commercial sources. All the synthesized oligonucleotides were desalted, and their concentrations were measured by absorbance at 260 nm.

2. PCR amplification and sequencing of genomic DNA. Genomic DNA was prepared from blood samples of 30 individuals employed by Los Alamos National Laboratory (LANL). All samples were obtained with informed consent as approved by the LANL Institutional Review Board. These samples had been previously sequenced using an automated DNA sequencer (PE Applied Biosystems, Foster City, Calif.) using standard methods. PCR amplification of an HLA-DBP1 exon II 320-bp fragment containing the Glu-69 SNP target was performed using the primers UG19 and UG21 described in Recheldi et al. (1993). Amplification of a 255-bp fragment from exon II of the HLA DPA1 gene used the primers described in Wang et al. (1999). Before minisequencing, the PCR-amplified template was treated with shrimp alkaline phosphatase (SAP, 1 unit, USB) and exonuclease I (Exol, 1 unit, USB) in SAP reaction buffer (USB) in a total volume of 10 ml at 37° C. for 1 h, followed by an inactivation step of 72° C. for 15 min. One microliter of the Exol/SAP-treated PCR product was used for each minisequencing reaction.

3. Preparation of microspheres. Streptavidin-coated and carboxylated microspheres (3.1 or 6.2 mm in diameter) were purchased from Spherotech, Inc. (Libertyville, Ill.). Avidin-coated or carboxylated multiplexing microspheres were purchased from Luminex Corp. (Austin, Tex.). In some cases, avidin (ExtraAvidin, Sigma, St. Louis, Mo.), or amino-bearing oligonucleotides were covalently attached to carboxylated microspheres using ethylenediaminocarbodiimide (EDAC, Pierce, Rockford, Ill.). Avidin (5 mg/ml) or amino-oligonucleotide (100 nM) and EDAC (10 mg/ml) were added, and the mixture was incubated for 30 min. Biotinylated oligonucleotides (100 nM) were bound to avidin- or streptavidin-coated microspheres (1 3 10⁷/ml) by incubation in TE buffer for at least 1 h at RT. The micropheres were washed by two cycles of centrifugation and resuspension to remove unbound oligonucleotides.

4. Capture/address tags. A random, insertion-deletion code (Varshamov and Tenengol'ts, 1965; Hazelwinkel, 1988, the teachings of which are hereby incorporated by reference herein), consisting of 1024 length-20 DNA sequences was designed. In this code, no subsequence common to any two code words contained more than 14 letters. These subsequences are not necessarily contiguous, and Needleman-Wunsch sequence alignment was used to find the length of the longest common subsequence, with matching letters contributing unity and mismatches and insertions/deletions contributing zero to the alignment score (Needleman and Wunsch, 1970, the teachings of which are hereby incorporated by reference herein). The rationale for implementing this code was that minimal cross-hybridization could occur between the reverse complement of one code word and another code word when the code words have only short subsequences in common. Sixteen of these code words were synthesized, see Table 1. This subset was derived from the code after further vetting with the Oligo program Molecular Biology Insights (Cascade, Colo.). The salient tests included duplex melting temperature, hairpin formation, matching to repetitive sequences in the DNA database, and cross-hybridization of capture tags.

5. Minisequencing assay. Minisequencing reactions were carried out in Thermosequenase buffer (Amersham Life Sciences, Cleveland, Ohio) in the presence of biotinylated or capture-tagged minisequencing primers (25 nM each), one FITC-labeled ddNTP (NEN/DuPont, Herts, UK), three nonfluorescent ddNTPs (5 mM each), Thermosequenase (1 unit, Amersham), and DNA template. The reaction was cycled 99 times at 94° C. for 10 s and at 60° C. for 10 s. After the minisequencing reaction, avidin- or address-tagged microspheres were added to each tube (5×10⁶) and incubated at room temperature for 1 h to capture the minisequencing primers. The hybridized bead mix was then diluted into 500 ml TE/BSA (50 mMTris-HCI, pH, 8.0, 0.5 mM EDTA, 0.5% (w/v) bovine serum albumin) for fluorescence measurement by flow cytometry.

6. Fluorescence detection by flow cytometry. Flow cytometric measurements of microsphere fluorescence were made on a Becton-Dickinson FACSCalibur (San Jose, Calif.) using CellQuest acquisition and analysis software. In some cases, multiplex samples were analyzed using the FlowMetrix O/R acquisition system (Luminex Corp.) interfaced with FACSCalibur. The samples were illuminated at 488 nm (15 mW), and forward-angle light scatter, 900 light scatter, and fluorescence signals were acquired. Linear amplifiers were used for all measurements. Particles were gated on forward angle and 900 light scatter, and the mean fluorescence channel numbers were recorded. The background fluorescence signal from unlabeled micro-spheres was subtracted from all samples. Mean fluorescence values were converted to mean equivalent soluble fluorophore units using Quantum 24 FITC Standard Microspheres from Flow Cytometry Standards Corp. (San Juan, Puerto Rico).

B. Results

A single biotinylated oligonucleotide annealed immediately adjacent to the SNP site is extended one base using DNA polymerase and fluorescent ddNTPs. The present assay configuration involves four parallel reactions, each with a different fluorescent ddNTP and three other nonfluorescent ddNTPs. The use of Thermosequenase, a thermostable DNA polymerase that efficiently incorporates ddNTPs, allows the minisequencing reactions to be cycled, thus amplifying the signal. After extension, the biotinylated primers were captured onto streptavidin- or avidin-coated microspheres, and the number of incorporated fluorescent ddNTPs was measured by flow cytometry.

TABLE 1
Multiplex Capture and Address Tag Sequences.

| Tag | Address | Capture |
|---|---|---|
| 1 | 5′TGAACCCGGGTATCTCACCA | 5′TGGTGAGATACCCGGGTTCA |
| 2 | 5′GGCTTTGGAGCGCTCTTTAA | 5′TTAAAGAGCGCTCCAAAGCC |
| 3 | 5′AGGAAAGGAGAGGCGTCGTC | 5′GACGACGCCTCTCCTTTCCT |
| 4 | 5′AACCACCTTAAGGGACGGAC | 5′GTCCGTCCCTTAAGGTGGTT |
| 5 | 5′GTACCCTCGGAAGGACCCAA | 5′TTGGGTCCTTCCGAGGGTAC |
| 6 | 5′AAAGTCGCGCCCAGAACCTC | 5′GAGGTTCTGGGCGCGACTTT |
| 7 | 5′TGTGTTCGGCGACTTGGTAG | 5′CTACCAAGTCGCCGAACACA |
| 8 | 5′ACCTGCTGGGCCGGGATGTT | 5′AACATCCCGGCCCAGCAGGT |
| 9 | 5′TTTCAGGTTCCACGGCATTG | 5′CAATGCCGTGGAACCTGAAA |
| 10 | 5′AAATGGCCTTGCTGTCTACG | 5′CGTAGACAGCAAGGCCATTT |
| 11 | 5′GTTCCGGTTrCGCCATGAGA | 5′TCTCATGGCGAAACCGGAAC |
| 12 | 5′ACGTGTTTCCCGCCAAATAT | 5′ATATTTGGCGGGAAACACGT |
| 13 | 5′GGCTGCTAAAGGCGTTCTAA | 5′TTAGAACGCCTTTAGCAGCC |
| 14 | 5′ATTAGGGTGCGCGCCATCTT | 5′AAGATGGCGCGCACCCTAAT |
| 15 | 5′CGAAGCATTTGGCCAATTTA | 5′TAAATTGGCCAAATGCTTCG |
| 16 | 5′CAGTTCGCCCAAAGGATAGG | 5′CCTATCCTTTGGGCGAACTG |

The polymorphism, amino acid position 69 in exon II of the HLA DPB1 locus was analyzed by the method of the present invention. This site is associated with immune hypersensitivity to the metal beryllium (Recheldi et al., 1993). A 320-bp fragment containing the site of interest was amplified from 30 different human genomic samples that had been sequenced previously, but had been coded to provide a “blind” test. A biotinylated minisequencing primer (18-mer) was designed to anneal immediately adjacent to this site. Four parallel reactions were set up containing the synthetic template, primer, polymerase, one of the four fluorescein-labeled ddNTPs, and the remaining three unlabeled ddNTPs. The reactions were cycled 99 times before the addition of the avidin capture beads. After capture of the primers, the samples were diluted 100-fold and analyzed by flow cytometry.

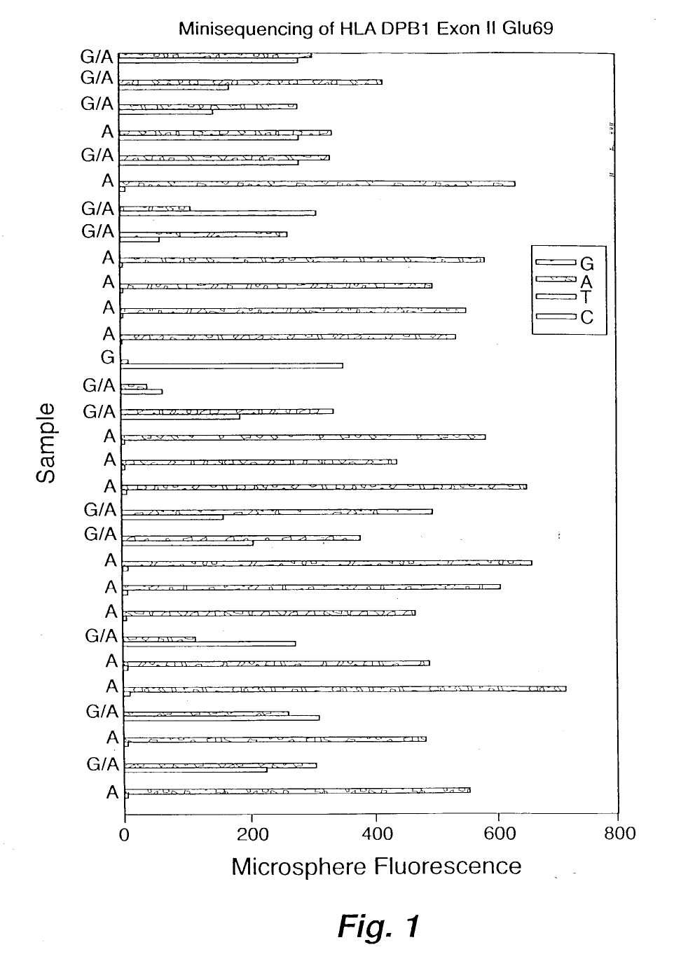

As shown in FIG. 1, the flow cytometric approach scored all 30 samples correctly, as judged by comparison to standard sequencing techniques, including 13 heterozygotes. These results were obtained without normalization of template concentration, which varied from sample to sample and ranged from approximately 1 to 0.1 nM. This variation likely accounts for some of the differences in the absolute signal amplitude observed among samples. Variation in signal intensities within samples for the hetereozygote results in part from differing fluorescence quantum yield of the fluorescent ddNTPs. In addition, sequence-specific effects, such as the differential amplification of particular alleles or variation in the minisequencing primer hybridization site, also likely contribute to this signal variation. Such factors are common to the minisequencing approach on any detection platform and do not impair the ability to determine base identity.

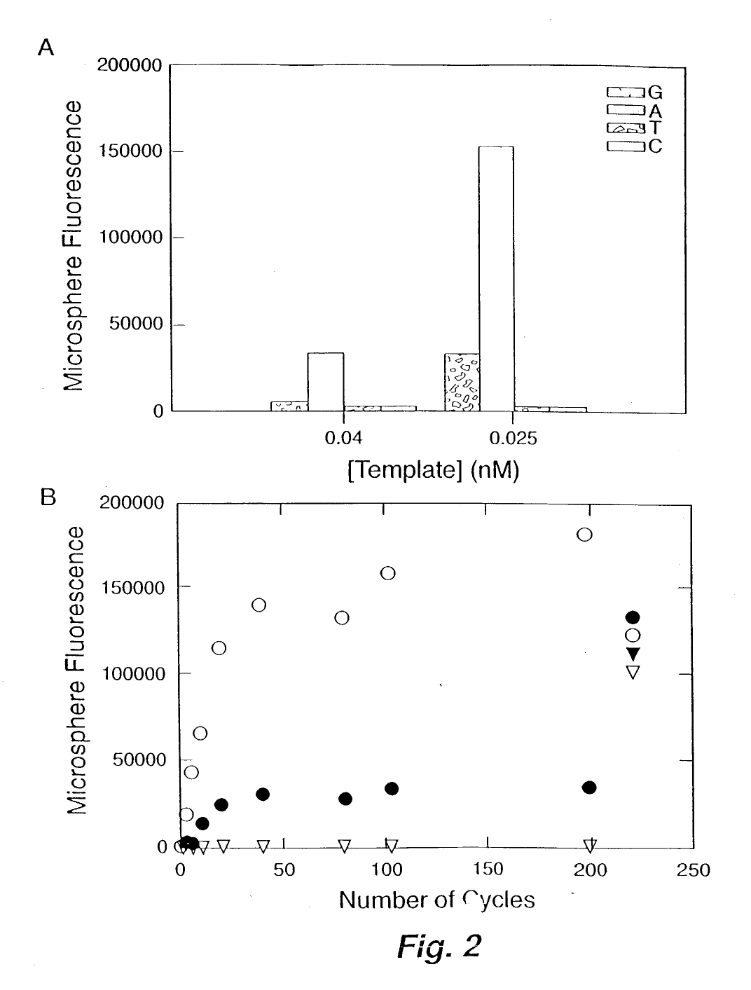

The ability to interrogate an individual template molecule with many primers through thermal cycling is important to the sensitivity of the minisequencing approach. Preliminary experiments indicated that maximal signal was achieved after between 50 and 100 cycles. Using 99 cycles, we determined that using ˜250 pM template (50 pg/ml of a 320-bp PCR product) allowed the genotype to be scored accurately (FIG. 2A). At 40 pM template, it was difficult to determine the genotype reliably under these cycling conditions. Often, however, the template is not limiting, especially with PCR-amplified template, and the speed of the assay is more important. Using a higher concentration of template (2 nM, or 0.4 ng/ml), enabled the accurate scoring of the genotype in as few as 10 cycles (FIG. 2B).

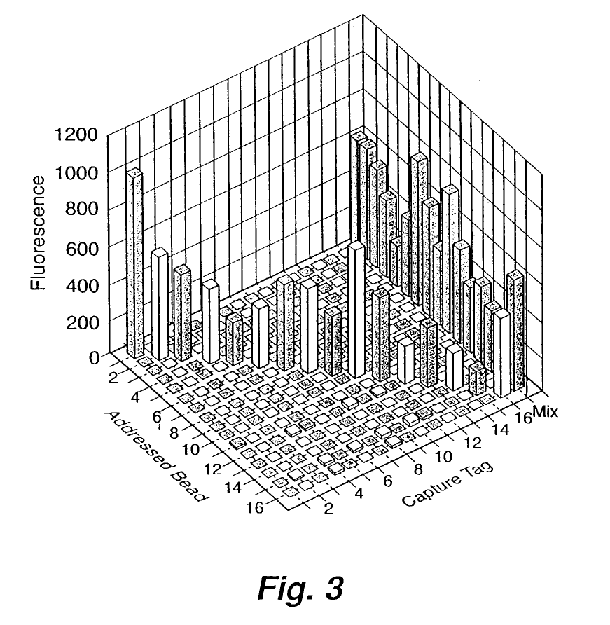

A key advantage of the flow cytometric method is the ability to perform multiplexed analyses using soluble arrays of differently stained microspheres (Fulton et al., 1997; Kettman et al., 1998). To adapt minisequencing to multiplexed microspheres, we first designed a set of address and capture oligonucleotide 20-mers. The sequences were designed to hybridize to only their respective complements and not to any other address or capture sequence. As presented in FIG. 3, when each address tag was attached to a specific microsphere subset, only the complementary fluorescent capture tag hybridized to the beads' surface, with negligible cross-talk. While there is some variability in fluorescence signal among the microsphere subpopulations, we have determined that this is due primarily to differences in the efficiency of modification of the biotin- or amino-modified synthetic oligonucleotides and that the signal of the dimmer beads can be increased simply increasing the concentration of the address tags during immobilization (data not presented). These results permit the implementation of a method where multiple MS primers, each bearing a unique 5′ capture sequence instead of a 5′ biotin, are captured onto address-tagged, rather than avidin-coated, microspheres.

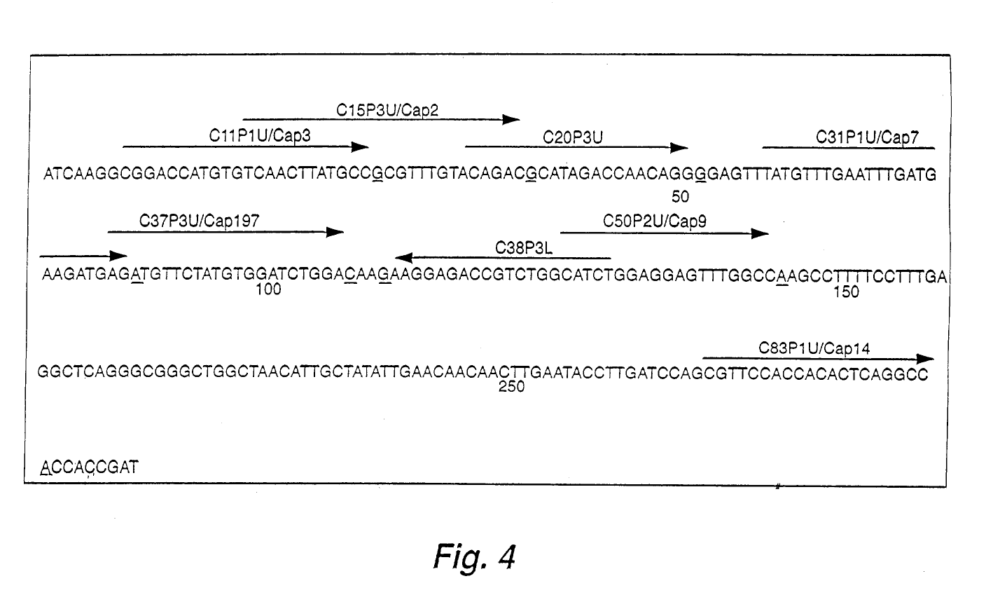

The multiplexed SNP scoring method of the present invention is demonstrated by genotyping common HLA DPA1 alleles. Variation in this region also appears to contribute to CBD susceptibility, especially in conjunction with the Glu69 allele (Wang et al., 1999). HLA alleles can be defined by the nucleotide base identity at several variable sites. For the alleles considered here (Table 2), there are eight SNP sites that can define alleles. Some of these sites are linked, so that a subset of the SNP sites can be used to identify individual alleles (Marsh and Bodmer, 1995). Minisequencing primers were designed to interrogate these eight SNPs, choosing a combination of Tm-matched upper and lower strand primers with the lowest tendency toward intramolecular hairpins and dimerization with themselves or any of the other primers. The close proximity of some SNP targets required a careful choice of primers to avoid competition for primer hybridization sites. For example, sites C37P3 and C38P3 are only three bases apart, necessitating the use of an upper strand primer to interrogate the first site and a lower strand primer for the second (FIG. 4). These primer sequences were then matched with 5′ capture tags from Table 1, again screening out undesirable interactions. Three of the eight minisequencing primers were not compatible with any of the 16 capture tags shown in Table 1. In one case (C37P3), a 17th capture/address pair was chosen from our capture/ad-dress database. In the other two cases, primer-specific address tags complementary to the primer sequences were used to capture these primers onto microspheres (see Table 3).

TABLE 2
DPA1 Exon 2 Allele-Defining SNPs.

| | C11 | C15 | C20 | C31 | C37 | C38 | C50 | C83 |
|---|---|---|---|---|---|---|---|---|
| DPA1 Allele | P1 | P3 | P3 | P1 | P3 | P3 | P2 | P1 |
| 01 | G | G | G | A | C | G | A | A |
| 02011 | G | C | G | C | T | A | G | G |
| 02012 | G | G | G | C | T | A | G | G |
| 02021 | A | C | A | C | T | G | G | G |
| 02022 | A | C | A | C | C | G | G | G |
| 0301 | A | C | G | A | C | G | A | A |
| 0401 | G | C | A | A | C | G | G | G |

Note.
C: codon number, P: position in a codon.

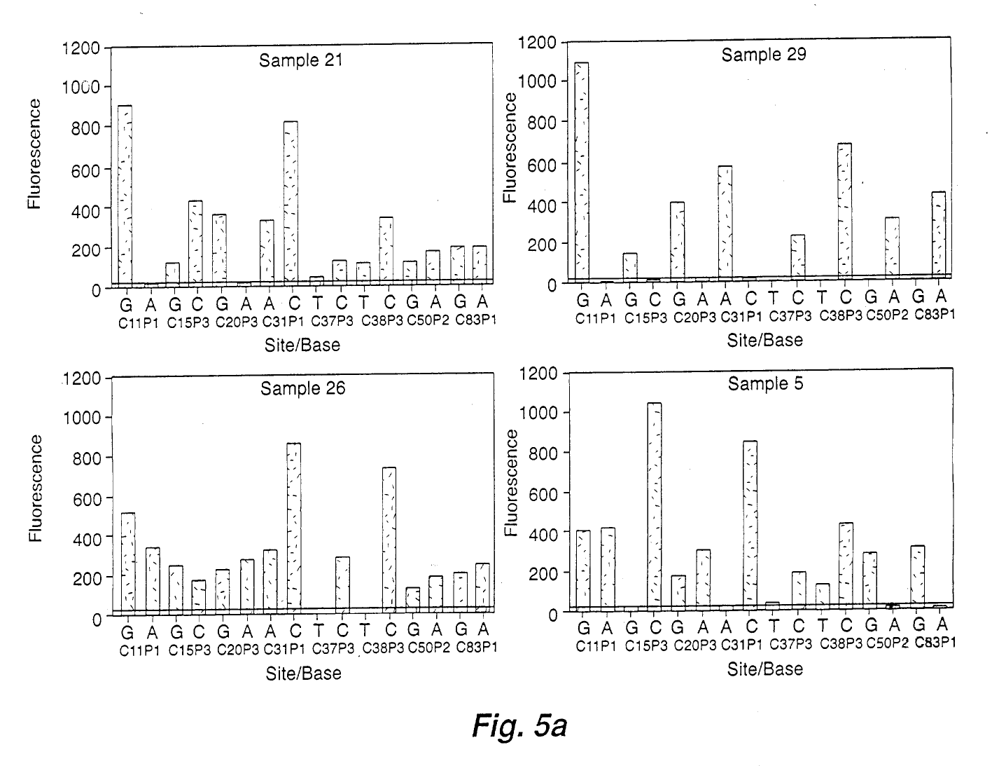

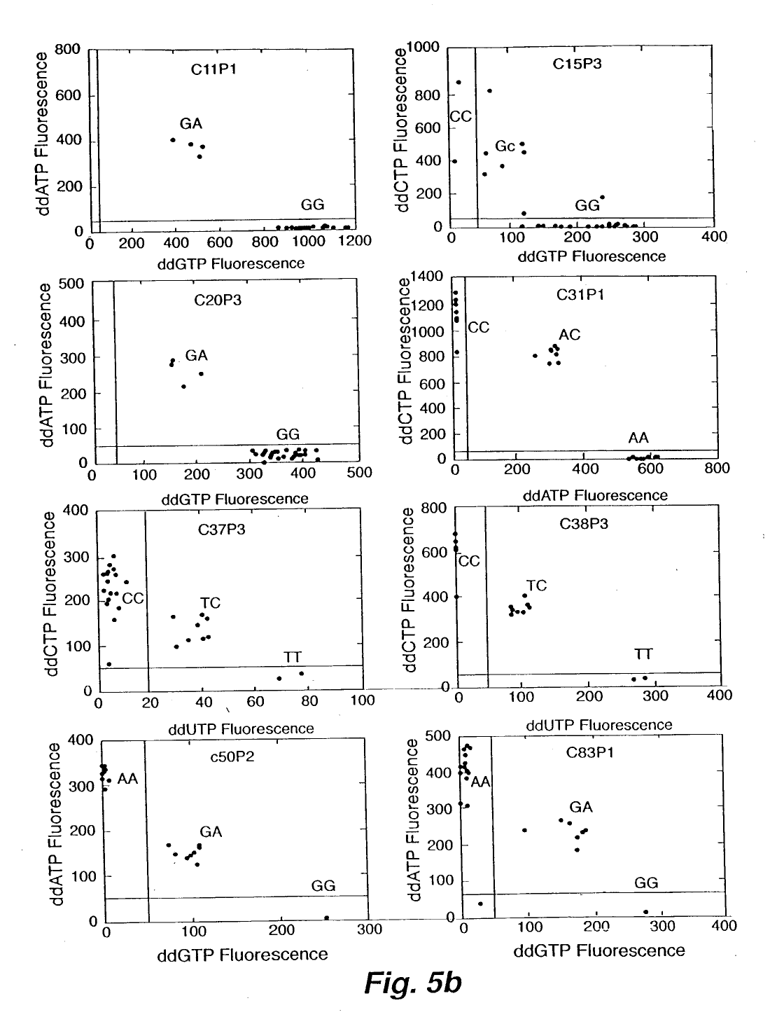

Presented in FIG. 5A are the multiplexed genotyping results at the eight biallelic DPA1 sites for four representative samples. The fluorescence values from incorporated bases ranged from approximately 10,000 to 100,000 MESF units per microsphere, with background signals ranging from 1000 to 5000 MESF units. In general, fluorescence signals from heterozygous samples were about half that from homozygous samples, consistent with a template concentration dependence for the minisequencing reaction. A threshold of 50 fluorescence units enabled positive base identification at all sites for all alleles except for T at site C37P3, which had lower signals overall and for which a threshold of 20 was used. By this method, the correct alleles for all 30 samples were identified (FIG. 5B, Table 4), as determined independently by direct DNA sequencing, representing the correct determination of nucleotide bases at eight sites on two chromosomes for each sample, or 480 sites total.

C. Discussion

The primer single-base extension method, also known as minisequencing, has been adapted to flow cytometry to enable multiplexed SNP analysis suitable for high-throughput applications. Using fluorescently stained microspheres bearing unique address tags, we were able to perform multiplexed primer extension with fluorescent ddNTPs on several SNPs simultaneously and subsequently capture primers onto microspheres for analysis by flow cytometry.

Flow-cytometry-based minisequencing has several advantages over other methods used for SNP scoring. First, because flow cytometry provides intrinsic resolution between free and particle-bound fluorophore, samples can be analyzed without any separation or wash steps. Second, flow cytometry is a very sensitive method of fluorescence detection. Most commercial instruments can easily measure a few thousand fluorescent molecules per particle. In the present assay, this sensitivity enables the analysis of DNA template at subnanomolar concentrations. Third, efficiency is improved by performing hybridization and primer extension in solution. Hybridization on a surface is much slower than hybridization in solution (Zammatteo et al., 1997). In preliminary experiments, it was found that minisequencing using an immobilized primer was much less efficient than with a soluble primer (data not presented). By performing hybridization and extension in solution, followed by capture on microspheres for analysis, the assay sensitivity and speed were further improved. Finally, because flow cytometry is a multiparameter detection platform, it is possible to mea-sure several features of a particle simultaneously. For example, it is possible to label each of the four ddNTPs with a different fluorophore, as is the case for dye-terminator sequencing, and detect them simultaneously in a single reaction.

TABLE 3
Sequences of HLA DPA1 Minisequencing Primers, Capture Tags, and Address Tags.

| Target | Capture sequence | Primer sequence | Address sequence |
|---|---|---|---|
| C11P1U/Cap3 | 5′GACGACGCCTCTCCTTTCCT | CGGACCATGTGTCAACTTATGCC | 5′AGGAAAGGAGAGGCGTCGTC |
| C15P3U/Cap2 | 5′TTAAAGAGCGCTCCAAAGCC | TCAACTTATGCCGCGTTTGTACAGAC | 5′GGCTTTGGAGCGCTCTTTAA |
| C20P3U | | 5′CAGACGCATAGACCAACAGG | 5′CCCTGTTGGTCTATGCGTCTG |
| C31P1U/Cap7 | 5′CTACCAAGTCGCCGMCACA | TATGTTTGAATTTGATGAAGATGAG | 5′TGTGTTCGGCGACTTGGTAG |
| C37P3U/Cap197 | 5′TCATGGCCCATGCGGGA | GAGATGTTCTATGTGGATCTGGA | 59TCCCGCATGGGCCATGA |
| C38P3L | | 5′CAGATGCCAGACGGTCTCCTT | 5′AAGGAGACCGTCTGGCATCTG |
| C50P2U/Cap9 | 5′TTTCAGGTTCCACGGCATTG | CATCTGGAGGAGTTTGGCC | 5′CAATGCCGTGGAACCTGAAA |
| C83P1U/Cap14 | 5′AAGATGGCGCGCACCCTAAT | CGTTCCAACCACACTCAGGCC | 5′AAGATGGCGCGCACCCTAAT |

Note. C: codon; P: position; Cap: capture probe; U or L: upper or lower primer.

The accuracy of the new genotyping method is conferred by the high fidelity of the DNA polymerase that fluorescently labels the capture-tagged primer. Minisequencing has been widely tested using a variety of detection platforms and has been found to be very robust (Syvanen, 1999). The design of multiplexed minisequencing assays requires considerations similar to those required for successful multiplex PCR, namely, avoiding primer heterodimers and false priming. Exon 2 of the DPA1 gene proved particularly challenging, because some of the allele-defining sites were close together (FIG. 2). This required careful choice of a combination of upper and lower strand primers, but resulted in the identification of the correct alleles in 30 of 30 samples. Some sites reproducibly gave high levels of signal (C11P1), while others gave low levels of signal (C37P3). The low-level signal at site C37P3 is most likely due to competition by the primer interrogating C31P1, a site that lies near the 5′-end of the C37P3 primer binding site. In most cases, the design of a lower strand primer to interrogate site C37P3 would have eliminated this complication. However, in this case site C38P3 lies near the 3′-end of the lower strand primer. Thus, the high density of variable sites in this exon restricts the placement of some primers. In addition, sequence variation in the primer hybridization site immediately 5′ of a SNP could account for the variable signal intensity observed between samples. These issues are common to multiplexed minisequencing in general, but they can often be overcome through careful primer design. Also, minisequencing does not reveal haplotype information, and definitive allele assignment will require the coupling of minisequencing to allele-specific PCR so that linkage can be determined.

Perhaps the most important advantage of the present flow-cytometry-based method is the ability to configure multiplexed SNP-scoring assays using soluble arrays of dyed microspheres. In this case, we performed a multiplexed analysis of the eight SNPs that define common alleles of the HLA DPA1 gene, another risk factor in chronic beryllium disease (Wang et al., 1999). The key to our implementation of the multiplexed analysis is the use of address-tagged microspheres and capture-tagged primers to target SNP-specific primers to identifiable microsphere subsets. As presented in FIG. 3, the set of 16 capture and address tags enabled the specific targeting of primers bearing fluorescent labels to individual microsphere subsets. The flow cytometer then measures and tabulates the fluorescence of each array element. The use of address-tagged microspheres as array elements provides a flexibility not possible with flat surface arrays. For example, our limited choice of minisequencing primers left us with 3 primers that were not compatible with any of the capture/address tag pairs in the original set of 16. Three new address tags were synthesized, immobilized on new aliquots of microspheres, and the DPA1 array was reconfigured with a pipette. In addition, the original 16-bead set was successfully used in genotyping applications ranging from bacterial strain identification to human mitochondrial DNA, with each new application requiring only the design and synthesis of primers attached to the appropriate capture tags. The concept of universal array addresses has previously been introduced for flat surface arrays (Gerry et al., 1999) and applied to microsphere arrays using sequences derived from the Mycobacterium tuberculosis genome (Iannone et al., 2000). The address tags used for the present invention represent a subset of more than 1000 sequences that were computationally designed to have the precise hybridization specificity and properties desired for multiplexed capture applications.

Sets of up to 100 dyed microspheres will soon be available commercially (Luminex Corp.). Each of the 100 microsphere subsets could be addressed to code for a unique primer, allowing the analysis of 100 SNPs in a single reaction. Because they can be readily prepared on the lab bench without any specialized equipment, microsphere arrays are much more flexible than two-dimensional microarrays on chips or slides. These features, combined with recent advances in automated sample handling (Nolan et al., 1995; Edwards et al., 1999), make flow cytometry an extremely attractive platform for high-throughput genotyping. In summary, a rapid and sensitive microsphere-based minisequencing assay has been developed for the multiplexed analysis of single nucleotide polymorphisms using flow cytometry. Incubations can be carried out in very small volumes (˜10 ml), subjected to thermocycling to amplify signal, and analyzed without a wash step at a rate of greater than one sample per minute. The optimal reaction conditions have been determined for the case where template is limited and sensitivity is most important as well as for the case where template is not limiting and speed is most important. Flow cytometers are widely available in core facilities in many universities and medical schools and in industry. The present invention makes it possible to rapidly screen large numbers of samples with a minimum of start-up costs and development time. Moreover, flow cytometry is also compatible with hybridization- and ligation-based assays (Fulton et al., 1997; Cai et al., 1998; Iannone et al., 2000), making it a versatile platform for a variety of genomic analyses.

TABLE 4
DPA1 Genotyping of 30 Human DNA Samples.

| Sample | DPA1 Allele/site | C11P1 | C15P3 | C20P3 | C31P1 | C37P3 | C38P3 | C50P2 | C83P1 |
|---|---|---|---|---|---|---|---|---|---|
| 1 | 01/01 | G | G | G | A | C | G | A | A |
| 2 | 02011/02022 | G/A | C | G/A | C | C/T | G/A | G | G |
| 3 | 01/02011 | G | G/C | G | A/C | C/T | G/A | G/A | G/A |
| 4 | 01/01 | G | G | G | A | C | G | A | A |
| 5 | 01/02012 | G | G | G | A/C | C/T | G/A | G/A | G/A |
| 6 | 01/01 | G | G | G | A | C | G | A | A |
| 7 | 01/02022 | G/A | G/C | G/A | A/C | C | G | G/A | G/A |
| 8 | 02011/02012 | G | G/C | G | C | T/C | A | G | G |
| 9 | 01/01 | G | G | G | A | C | G | A | A |
| 10 | 01/01 | G | G | G | A | C | G | A | A |
| 11 | 01/02011 | G | G/C | G | A/C | T/C | G/A | G/A | G/A |
| 12 | 01/01 | G | G | G | A | C | G | A | A |
| 13 | 01/01 | G | G | G | A | C | G | A | A |
| 14 | 01/01 | G | G | G | A | C | G | A | A |
| 15 | 01/02011 | G | G/C | G | A/C | T/C | G/A | G/A | G/A |
| 16 | 01/01 | G | G | G | A | C | G | A | A |
| 17 | 01/01 | G | G | G | A | C | G | A | A |
| 18 | 01/01 | G | G | G | A | C | G | A | A |
| 19 | 01/01 | G | G | G | A | C | G | A | A |
| 20 | 01/01 | G | G | G | A | C | G | A | A |
| 21 | 01/01 | G | G | G | A | C | G | A | A |
| 22 | 01/01 | G | G | G | A | C | G | A | A |
| 23 | 2012/2012 | G | G | G | C | T | A | A | G |
| 24 | 01/02012 | G | G | G | A/C | T/C | G/A | G/A | G/A |
| 25 | 01/01 | G | G | G | A | C | G | A | A |
| 26 | 01/02011 | G | G/C | G | A/C | T/C | G/A | G/A | G/A |
| 27 | 01/01 | G | G | G | A | C | G | A | A |
| 28 | 01/02022 | G/A | G/C | G/A | A/C | C | G | G/A | G/A |
| 29 | 02011/02022 | G/A | C | G/A | C | T/C | G/A | G | G |
| 30 | 01/01 | G | G | G | A | C | G | A | A |

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

### REFERENCES

Braun, A., Little, D. P., and Koster, H. (1997). Detecting CFTR gene mutations by using primer oligo base extension and mass spectrometry. Clin. Chem. 43: 1151-1158.

Brookes, A. J. (1999). The essence of SNPs. Gene 234: 177-186.

Cai, H., Kommander, K., White, P. S., Keller, R., and Nolan, J. P. (1998). Flow cytometry-based hybridization and polymorphism detection and analysis, In “Advances in Optical Biophysics” (J. R. Lakowicz and J. B. A. Ross, Eds.), Proceedings of the SPIE, Vol. 3256, pp. 3171-3177.

Chen, X. N., and Kwok, P. Y. (1999). Homogeneous genotyping assays for single nucleotide polymorphisms with fluorescence resonance energy transfer detection. Genet. Anal. Biomol. Eng. 14:157-163.

Cooper, D. N., and Krawczak, M. (1990). The mutational spectrum of single base-pair substitutions causing human genetic disease: Patterns and predictions. Hum. Genet. 85: 55-74.

Cooper, D. N., Smith, B. A., Cook, H. J., Hiemann, S., and Schmidtke, J. (1985). An estimate of unique DNA sequence heterozygosity in the human genome. Hum. Genet. 69: 201-205.

Edwards, B. S., Kuckuck, F., and Sklar, L. A. (1999). Plug flow cytometry: An automated coupling device for rapid sequential flow cytometric sample analysis. Cytometry 37: 156-159.

Fulton, R. J., McDade, R. L., Smith, P. L., Kienker, L. J., and Kettman, J. R. (1997). Advanced multiplexed analysis with the Flowmetrix system. Clin. Chem. 43: 1749-1756.

Gerry, N. P., Witkowski, N. E., Day, J., Hammer, R. P., Barany, G., and Barany, F. (1999). Universal DNA microarray method for multiplex detection of low abundance point mutations. J. Mol. Biol. 292: 251-262.

Hazelwinkel, M. (1988). “Encyclopaedia of Mathematics,” Kluwer, Dordrecht.

Iannone, M. A., Taylor, J. D., Chen, J., Li, M. S., Rivers, P., Slentz-Kesler, K. A., and Weiner, M. P. (2000). Multiplexed single nucleotide polymorphism genotyping by oligonucleotide ligation and flow cytometry. Cytometry 39: 131-140.

Kaderali, L. Selecting Target Specific Probes for DNA Arrays, Master's Thesis, Informatics, U. K\″{o}ln, (2001).

Keftman, J. R., Davies, T., Chandler, D., Oliver, K. G., and Fulton, R. J. (1998). Classification and properties of 64 multiplexed microsphere sets. Cytometry 33: 234-243.

Landegren, U., Kaiser, R., Sanders, J., and Hood, L. (1988). A ligase-mediated gene detection technique. Science 24:1077-1080.

Lee, L. G., Connell, C. R., and Bloch, W. (1993). Allelic discrimination by nick-translation PCR with fluorogenic probes. Nucleic Acids Res. 21: 3761-3766.

Lyamichev, V., Mast, A. L., Hall, J. G., Prudent, J. R., Kaiser, M. W., Takova, T., Kwiatkowski, R. W., Sander, T. J., deArruda, M., Arco, D. A., Neri, B. P., and Brow, M. A. D. (1999). Polymorphism identification and quantitative detection of genomic DNA by invasive cleavage of oligonucleotide probes. Nat. Biotechnol. 17: 292-296.

Marsh, S. G. E., and Bodmer, J. G. (1995). HLA class 11 region nucleotide sequences. Tissue Antigens 45: 258-280.

Needleman, S. B., and Wunsch, C. D. (1970). A general method applicable to the search for similarities in the amino-acid sequences of two proteins. J. Mol. Biol. 48: 443-453.

Nolan, J. P., Posner, R. G., Habberseft, R. C., Martin, J. C., and Sklar, L. A. (1995). A rapid mix flow cytometer with subsecond kinetic resolution. Cytometry 21: 223-229.

Nolan, J. P., and Sklar, L. A. (1998). The emergence of flow cytometry for sensitive, real-time analysis of molecular interactions. Nat. Biotechnol. 16: 633-638. Pastinen, T., Kurg, A., Metspalu, A., Peltonen, L., and Syvanen, A. C. (1997). Minisequencing: A specific tool for DNA analysis and diagnostics on oligonucleotide arrays. Genome Research 7: 606-614.

Pastinen, T., Partanen, J., and Syvanen, A. C. (1996). Multiplex, fluorescent, solid-phase minisequencing for efficient screening of DNA sequence variation. Clin. Chem. 42:1391-1397.

Recheldi, L., Sorrentino, R., and Saltini, C. (1993). HLA-DPB1 glutamate-69: A genetic marker of beryllium disease. Science 262: 242-244.

Schaffer, A. J., and Hawkins, J. R. (1998). DNA variation and the future of human genetics. Nat. Biotechnol. 16: 33-39.

Shumaker, J. M., Metspalu, A., and Caskey, C. T. (1996). Mutation detection by solid-phase primer extension. Hum. Mutat. 7: 346-354.

Syvanen, A., Aalto-Setala, K., Kontula, K., and Soderlund, H. (1990). A primer-guided nucleotide incorporation assay in genotyping of apolipoprotein E. Genomics 8: 684-692.

Syvanen, A. C. (1999). From gels to chips: “Minisequencing” primer extension for analysis of point mutations and single nucleotide polymorphisms. Hum. Mutat. 13: 1-10.

Tobe, V. O., Taylor, S. L., and Nickerson, D. A. (1996). Single-well genotyping of diallelic sequence variations by a 2-color ELISA-based oligonucleotide ligation assay. Nucleic Acids Res. 24: 3728-3732.

Tully, G., Sullivan, K. M., Nixon, P., Stones, R. E., and Gill, P. (1996). Rapid detection of mitochondrial sequence polymorphisms using multiplex solid-phase fluorescent minisequencing. Genomics 34:107-113.

Varshamov, R. R., and Tenengol'ts, G. M. (1965). One-asymmetrical-error correction codes. Avtomatika i Telemekhanika 26: 288-292.

Wang, D. G., Fan, J. B., Siao, C. J., Berno, A., Young, P., Sapolsky, R., Ghandour, G., Perkins, N., Winchester, E., Spencer, J., Kruglyak, L., Stein, L., Hsie, L., Topaloglou, T., Hubbell, E., Robinson, E., Mittmann, M., Morris, M. S., Shen, N. P., Kilburn, D., Rioux, J., Nusbaum, C., Rozen, S., Hudson, T. J., Lipshutz, R., Chee, M., and Lander, E. S. (1998). Large-scale identification, mapping, and genotyping of single nucleotide polymorphisms in the human genome. Science 280: 1077-1082.

Wang, Z., White, P. S., Petrovic, M., Tatum, 0. L., Newman, L. S., Maier, L. A., and Marrone, B. L. (1999). Differential susceptibilities to chronic beryllium disease contributed by different Glu 69 HLA-DPB1 and DPA1 alleles. J. Immunol. 163: 1647-1653.

Zammatteo, N., Alexandre, I., Ernest, I., Le, L., Brancart, F., and Remacle, J. (1997). Comparison between microwell and bead sup-ports for the detection of cytomegalovirus amplicons by sandwich hybridization. Anal. Biochem. 253: 180-189.

What is claimed is:

1. A method for identifying a set of sequences useful as address/capture tags which comprises the steps of:
(a) generating a chosen number of single-stranded, random oligonucleotide sequences having a chosen length;
(b) rejecting all sequences from said chosen number of single-stranded, random oligonucleotide sequences having common subsequences with a subsequence length greater than a chosen number of bases, the remaining sequences forming a first group of sequences;
(c) rejecting all sequences in said first group of sequences which can form stable hairpins, the remaining sequences forming a second group of sequences; and
(d) rejecting all sequences in said second group of sequences which can form stable dimers, the remaining sequences forming a third group of sequences; whereby a set of sequences is identified such that the sequences, if synthesized, would hybridize to their respective complements with a high degree of specificity.

2. The method for identifying a set of sequences useful as address/capture tags as described in claim 1, further comprising the step rejecting all reverse complementary sequences from said third group of sequences, the remaining sequences forming a fourth group of sequences;

3. The method for identifying a set of sequences useful as address/capture tags as described in claim 2, further comprising the steps of determining the melting temperature of each of sequence in said fourth group of sequences; and rejecting all sequences that melt below a selected temperature, forming thereby a fifth group of sequences.

4. The method as described in claim 3, further comprising the steps of synthesizing a desired number of the sequences in the fifth group of sequences, and synthesizing the complements thereof.

5. The method for generating a set of address/capture tags as described in claim 3, wherein said selected melting temperature is between 50° C. and 70° C.

6. The method for generating a set of address/capture tags as described in claim 5, wherein said selected melting temperature is about 60° C.

7. The method for generating a set of address/capture tags as described in claim 1, further comprising the step of rejecting all runs of bases greater than a chosen number of bases.

8. The method for generating a set of address/capture tags as described in claim 7, wherein the chosen number of bases is 2.

9. The method for generating a set of address/capture tags as described in claim 1, wherein said chosen number of random DNA sequences is computationally generated.

10. The method for generating a set of address/capture tags as described in claim 4, wherein said synthesized sequences are immobilized on identifiable microparticles, each of said synthesized sequences being immobilized on a different identifiable microsphere.

11. The method for generating a set of address/capture tags as described in claim 4, wherein said synthesized complementary sequences are immobilized on identifiable microparticles, each of said synthesized complementary sequences being immobilized on a different identifiable microsphere.

12. The method for generating a set of address/capture tags as described in claim 4, wherein the address/capture tags are used for multiplexed SNP scoring in a flow cytometer assay.

13. A method for generating a set of address/capture tags which comprises the steps of:
(a) generating a chosen number of single-stranded, random oligonucleotide sequences having a chosen length;
(b) rejecting all reverse complementary sequences from said chosen number of random oligonucleotide sequences, the remaining sequences forming a first group of sequences;
(c) rejecting all sequences having runs of bases greater than a chosen number of bases, the remaining bases forming a second group of bases;
(d) rejecting all sequences from said second group of sequences having common subsequences with a subsequence length greater than a chosen number of bases, the remaining sequences forming a third group of sequences;
(e) rejecting all sequences in said third group of sequences which can form stable hairpins, the remaining sequences forming a fourth group of sequences;
(f) rejecting all sequences in said fourth group of sequences which can form stable dimers, the remaining sequences forming a fifth group of sequences;
(g) determining the melting temperature of each of sequence in said fifth group of sequences;
(h) rejecting all sequences that melt below a selected temperature, forming thereby a sixth group of sequences;
(i) synthesizing a desired number of the sequences in said sixth group of sequences; and
(j) synthesizing the complementary sequences of said desired number of sequences, whereby a set of address/capture tags is generated such that the synthesized sequences hybridize to their respective complementary sequences with a high degree of specificity.


SEQUENCE LISTING (55 sequences)

SEQ ID NO: 1 - DNA, 20 bp, unknown
  Address tag
tgaacccgggtatctcacca

SEQ ID NO: 2 - DNA, 20 bp, unknown
  Capture tag
tggtgagatacccgggttca

SEQ ID NO: 3 - DNA, 20 bp, Unknown
  Address tag
ggctttggagcgctctttaa

SEQ ID NO: 4 - DNA, 20 bp, Unknown
  Capture tag
ttaaagagcgctccaaagcc

SEQ ID NO: 5 - DNA, 20 bp, Unknown
  Address tag
aggaaaggagaggcgtcgtc

SEQ ID NO: 6 - DNA, 20 bp, Unknown
  Capture tag
gacgacgcctctcctttcct

SEQ ID NO: 7 - DNA, 20 bp, Unknown
  Address tag
aaccaccttaagggacggac

SEQ ID NO: 8 - DNA, 20 bp, Unknown
  Capture tag
gtccgtcccttaaggtggtt

SEQ ID NO: 9 - DNA, 20 bp, Unknown
  Address tag
gtaccctcggaaggacccaa

SEQ ID NO: 10 - DNA, 20 bp, Unknown
  Capture tag
ttgggtccttccgagggtac

SEQ ID NO: 11 - DNA, 20 bp, Unknown
  Address tag
aaagtcgcgcccagaacctc

SEQ ID NO: 12 - DNA, 20 bp, Unknown
  Capture tag
gaggttctgggcgcgacttt

SEQ ID NO: 13 - DNA, 20 bp, Unknown
  Address tag
tgtgttcggcgacttggtag

SEQ ID NO: 14 - DNA, 20 bp, Unknown
  Capture tag
ctaccaagtcgccgaacaca

SEQ ID NO: 15 - DNA, 20 bp, Unknown
  Address tag
acctgctgggccgggatgtt

SEQ ID NO: 16 - DNA, 20 bp, Unknown
  Capture tag
aacatcccggcccagcaggt

SEQ ID NO: 17 - DNA, 20 bp, Unknown
  Address tag
tttcaggttccacggcattg

SEQ ID NO: 18 - DNA, 20 bp, Unknown
  Capture tag
caatgccgtggaacctgaaa

SEQ ID NO: 19 - DNA, 20 bp, Unknown
  Address tag
aaatggccttgctgtctacg

SEQ ID NO: 20 - DNA, 20 bp, Unknown
  Capture tag
cgtagacagcaaggccattt

SEQ ID NO: 21 - DNA, 20 bp, Unknown
  Address tag
gttccggtttcgccatgaga

SEQ ID NO: 22 - DNA, 20 bp, Unknown
  Capture tag
tctcatggcgaaaccggaac

SEQ ID NO: 23 - DNA, 20 bp, Unknown
  Address tag
acgtgtttcccgccaaatat

SEQ ID NO: 24 - DNA, 20 bp, Unknown
  Capture tag
atatttggcgggaaacacgt

SEQ ID NO: 25 - DNA, 20 bp, Unknown
  Address tag
ggctgctaaaggcgttctaa

SEQ ID NO: 26 - DNA, 20 bp, Unknown
  Capture tag
ttagaacgcctttagcagcc

SEQ ID NO: 27 - DNA, 20 bp, Unknown
  Address tag
attagggtgcgcgccatctt

SEQ ID NO: 28 - DNA, 20 bp, Unknown
  Capture tag
aagatggcgcgcaccctaat

SEQ ID NO: 29 - DNA, 20 bp, Unknown
  Address tag
cgaagcatttggccaattta

SEQ ID NO: 30 - DNA, 20 bp, Unknown
  Capture tag
taaattggccaaatgcttcg

SEQ ID NO: 31 - DNA, 20 bp, Unknown
  Address tag
cagttcgcccaaaggatagg

SEQ ID NO: 32 - DNA, 20 bp, Unknown
  Capture tag
cctatcctttgggcgaactg

SEQ ID NO: 33 - DNA, 20 bp, Unknown
  Capture sequence
gacgacgcctctcctttcct

SEQ ID NO: 34 - DNA, 23 bp, Unknown
  Primer sequence
cggaccatgtgtcaacttatgcc

SEQ ID NO: 35 - DNA, 20 bp, Unknown
  Address sequence
aggaaaggagaggcgtcgtc

SEQ ID NO: 36 - DNA, 20 bp, Unknown
  Capture sequence
ttaaagagcgctccaaagcc

SEQ ID NO: 37 - DNA, 26 bp, Unknown
  Primer sequence
tcaacttatgccgcgtttgtacagac

SEQ ID NO: 38 - DNA, 20 bp, Unknown
  Address sequence
ggctttggagcgctctttaa

SEQ ID NO: 39 - DNA, 20 bp, Unknown
  Primer sequence
cagacgcatagaccaacagg

SEQ ID NO: 40 - DNA, 21 bp, Unknown
  Address sequence
ccctgttggtctatgcgtctg

SEQ ID NO: 41 - DNA, 20 bp, Unknown
  Capture sequence
ctaccaagtcgccgaacaca

SEQ ID NO: 42 - DNA, 25 bp, Unknown
  Primer sequence
tatgtttgaatttgatgaagatgag

SEQ ID NO: 43 - DNA, 20 bp, Unknown
  Address sequence
tgtgttcggcgacttggtag

SEQ ID NO: 44 - DNA, 17 bp, Unknown
  Capture sequence
tcatggcccatgcggga

SEQ ID NO: 45 - DNA, 23 bp, Unknown
  Primer sequence
gagatgttctatgtggatctgga

SEQ ID NO: 46 - DNA, 17 bp, Unknown
  Address sequence
tcccgcatgggccatga

SEQ ID NO: 47 - DNA, 21 bp, Unknown
  Primer sequence
cagatgccagacggtctcctt

SEQ ID NO: 48 - DNA, 21 bp, Unknown
  Address sequence
aaggagaccgtctggcatctg

SEQ ID NO: 49 - DNA, 20 bp, Unknown
  Capture sequence
tttcaggttccacggcattg

SEQ ID NO: 50 - DNA, 19 bp, Unknown
  Primer sequence
catctggaggagtttggcc

SEQ ID NO: 51 - DNA, 20 bp, Unknown
  Address sequence
caatgccgtggaacctgaaa

SEQ ID NO: 52 - DNA, 20 bp, Unknown
  Capture sequence
aagatggcgcgcaccctaat

SEQ ID NO: 53 - DNA, 21 bp, Unknown
  Primer sequence
cgttccaaccacactcaggcc

SEQ ID NO: 54 - DNA, 20 bp, Unknown
  Address sequence
aagatggcgcgcaccctaat

SEQ ID NO: 55 - DNA, 254 bp, Human HLA
atcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaccacactcaggccaccaccgat